(19) United States
(12) Patent Application Publication
(10) Pub. No.: US 2002/0193649 A1
(43) Pub. Date: Dec. 19, 2002

(54) Synthesis of high quality normal alpha olefins

(76) Inventors: Dennis J. O'Rear, Petaluma, CA (US); William L. Schinski, San Rafael, CA (US); Saleh Elomari, Richmond, CA (US); Richard N. Reynolds, Point Richmond, CA (US); Steven J. Herron, Houston, TX (US)

Correspondence Address: CHEVRON PHILLIPS CHEMICAL COMPANY LP, LAW DEPARTMENT - IP, P.O BOX 4910, THE WOODLANDS, TX, 77387-4910, US

(21) Appl. No.: 10/040,822

(22) Filed: Jan. 7, 2002

Related U.S. Application Data
(63) Continuation of application No. 09/608,261, filed on Jun. 30, 2000, now abandoned.

(51) Int. Cl. C07C 11/00; C07C 9/00

(52) U.S. Cl. 585/324; 585/330; 585/16

(57) ABSTRACT

Processes for converting linear hydrocarbon into normal alpha olefins (“NAOs”). The process comprises a sequence of controlled dehydrogenation followed by ethenolysis. The process is especially applicable to upgrade linear hydrocarbons such as produced by Fischer-Tropsch type processes. A process for converting C₁-C₃ alkanes into NAOs and an integrated process including hydrocracking are also disclosed.

### BACKGROUND OF THE INVENTION

This invention relates to the conversion of linear hydrocarbons into normal alpha olefins (NAOs) via a process involving the dehydrogenation of linear paraffins into internal olefins and the conversion of the internal olefins into NAOs. Because of their linearity the invention is especially applicable to feedstocks prepared by Fischer-Tropsch type processes. In a further aspect the invention relates to the conversion of C₁-C₃ alkane rich gases to NAOs and other useful liquid hydrocarbons such as lubricating oil and liquid fuels. (The term liquid fuels refers to hydrocarbons used as fuels, for example, gasoline, diesel oil, jet fuel, kerosene, and the like, which are liquid at ambient conditions, including however, pentane.)

In general NAOs are made by three different routes i.e., thermal cracking of linear paraffins, oligomerization of ethylene, and a modified ethylene oligomerization process. In the modified process excess NAOs produced by the oligomerization process are converted to internal olefins either by isomerization or reaction over a metathesis catalyst. The internal olefins are then mixed with an excess of ethylene and passed over a metathesis catalyst converting the internal olefin into the desired NAOs. The prior art thermal cracking route suffers from the disadvantage of low product purity and the production of dienes. The two oligomerization processes suffer from the disadvantage of requiring an expensive feedstock (i.e. ethylene) and are directly based on ethylene; i.e. stoichiometrically one mole of a CₙNAO requires ½n moles of ethylene.

The production of α-olefins by the disproportionation or ethenolysis of internal olefins is described in U.S. Pat. No. 3,647,906; Crain et al., “Synthesis of Olefins via Disproportionation,” ACS DIV PET CHEM PREPR V17 N.4 (1972) p. E80-E85; J. C. Mol and J. A. Moulijn and “Catalytic Metathesis of Alkenes,” Catalysis Science and Technology edited by John R. Anderson and Michel Boudart. Vol. 8, Chapter 2. Conversely a process for converting NAOs into beta olefins and internal monoolefins is described in U.S. Pat. No. 3,776,974. Processes for dehydrogenating hydrocarbons are known and are for example described in U.S. Pat. Nos. 4,046,715 and 4,124,649.

In one aspect of the invention, we have now developed an economical process for converting linear hydrocarbons to high purity C₆ and higher NAOs.

### SUMMARY OF THE INVENTION

The present invention provides processes for converting linear hydrocarbons having ten or more carbon atoms into C₆ and higher NAOs. In one aspect the invention provides a process for converting C₁₀ and higher linear hydrocarbon into C₆ and higher NAOs which process comprises the steps of:

a) dehydrogenating a hydrocarbon mixture comprising at least 50 wt. % C₁₀ and higher linear paraffinic compounds to produce linear internal olefins and

b) contacting said linear internal olefins with ethylene in the presence of an ethenolysis catalyst under ethenolysis reactive conditions thereby producing C₆ and higher NAOs.

The invention also provides integrated processes comprising the aforedescribed process and feedstock purification and/or hydrogenation process steps as well as collateral operations resulting in the preparation of liquid fuels and/or lubricating oils as well as the NAOs. As used herein the term “linear” paraffinic compounds” or “linear paraffinic hydrocarbons” excludes olefins but includes linear paraffins and other linear paraffinic compounds such as non-olefinic linear oxygenates, e.g., linear paraffinic alcohols and linear paraffinic acids, e.g., saturated fatty acids.

In another embodiment the invention provides a process for converting hydrocarbon gases containing at least 50 wt. % methane, ethane, or propane or mixtures thereof (hereafter referred to as C₁-C₃ alkanes) to NAOs which comprises:

a) reforming C₁-C₃ alkanes into synthesis gas for example by steam reforming, partial oxidation or catalytic oxidation;

b) converting said synthesis gas into a linear hydrocarbon product comprising at least about 20 wt. % C₁₀ and higher linear paraffinic hydrocarbons and recovering the C₁₀ and higher linear hydrocarbons;

c) hydrotreating said recovered higher linear paraffinic hydrocarbons to convert linear oxygenates and linear olefins into linear paraffins thereby yielding a hydrogenated reaction product comprising at least 70 wt. % and preferably at least 90 wt. % C₁₀ and higher linear paraffins;

d) dehydrogenating the hydrocarbon reaction product of step (c) or a selected fraction thereof to produce the corresponding internal olefins;

e) contacting said internal olefins with ethylene in the presence of an ethenolysis catalyst under ethenolysis reactive conditions thereby producing NAOs and recovering the desired carbon atom range NAOs and recycling back to the hydrotreating step or the dehydrogenation step any NAOs, paraffins and internal olefins boiling higher than said desired carbon atom range NAOs.

The present invention further provides an integrated process for converting hydrocarbon gases containing at least 50 wt. % methane, ethane or propane or mixtures thereof (hereinafter referred to C₁-C₃ alkanes) into NAOs and higher molecular weight liquid fuels (C₅ and higher) and/or lubricating base oils which comprises the steps of:

a) reforming said C₁-C₃ alkanes into synthesis gas for example, by steam reforming, partial oxidation or catalytic oxidation;

b) contacting the synthesis gas with a Fischer-Tropsch catalyst under reactive conditions to yield two hydrocarbon product streams, one a wax containing product boiling above about 350° F.-800° F. (177° C.-427° C.) and typically above about 350° F.-700° F. depending on the molecular weight of the wax cut selected comprising vacuum gas oil through heavy paraffins including at least about 20 wt. % of the selected wax cut in the range of C₁₀-C₂₄ and higher linear paraffinic hydrocarbons and a second product boiling below about the selected wax cut (i.e., below the selected value in the range of 350° F.-800° F.), comprising hydrocarbons boiling in the gasoline through middle distillate range including diesel fuel (liquid fuel range) depending on the cut taken range and tail gases;

c) fractionating the wax containing product of step (b) into fractions comprising a paraffinic hydrocarbon fraction in the range of from a selected n-paraffin in the range of C₁₀ to C₂₈ at the lower end to about a C₅₀ paraffin at the upper end boiling in about the range of the select value to about 1100° F. (593° C.), or preferably a C₁₆ to C₅₀ paraffin fraction boiling in the range of about 550° F. (288° C.) to about 1100° F., containing at least about 70 wt. % and preferably at least about 90 wt. % C₁₆ to C₅₀ linear paraffinic hydrocarbons, a liquid fuel fraction boiling below about the n-paraffin before selected and a heavy fraction boiling above about 1100° F. (593° C.);

d) dehydrogenating the paraffinic hydrocarbon fraction of step (a) to produce linear internal olefins;

e) contacting said linear internal olefins with ethylene in the presence of an ethenolysis catalyst under reactive conditions thereby producing a reaction product comprising a substantial amount of C₆-C₂₄ NAOs;

f) fractionating the reaction product of step (e) into an ethylene fraction including ethylene and hydrocarbons having less than six carbon atoms, a NAO product fractions of varying chain length within the range of C₆-C₂₄, a higher boiling fraction comprising hydrocarbons having more than 24 carbon atoms internal olefins and optionally purifying the C₆-C₂₄ NAO fraction to remove C₆-C₂₄ paraffins and internal olefins;

g) hydrotreating or hydroisomerizing at least a portion of at least one of the liquid fuel portion of the second product of step (b); the heavy fraction of step (c) and/or the higher boiling fraction of step (f) and optionally hydrocracking at least a portion of the heavy fraction of step (c) and/or at least a portion of the fraction of the heavy fraction of step (f) boiling above about 700° F.; and

h) optionally fractionating any reaction products of step (g) having a substantial portion of hydrocarbon product boiling above and below about 700° F. and recovering at least one liquid fuel fraction boiling below about 700° F., and at least one higher boiling hydrocarbon fraction and recycling at least one of said higher boiling hydrocarbon fractions back to said hydrocracker.

In another embodiment the invention provides a process for upgrading a substantially full boiling range Fischer-Tropsch reaction product including tail gases through hydrocarbons boiling above 1100° F., e.g., bright stock.

The invention further provides a novel NAO product mixture having improved properties, especially lower levels of diolefin, saturates, vinylidines in comparison to products made by conventional routes.

Additional aspects of the invention will be apparent from the description which follows:

### BRIEF DESCRIPTION OF THE DRAWING

FIG. 1 is a schematic flow sheet of an embodiment of the invention for converting C₁₀ and higher linear hydrocarbon feed stocks derived from petroleum to C₆ and higher NAOs.

FIG. 2 is a schematic flow sheet of an integrated process according to the invention for converting natural gas to C₆-C₂₄ NAOs and liquid fuels and/or lube base oils.

### FURTHER DESCRIPTION OF THE INVENTION

The present invention provides an efficient process for preparing C₆ and higher NAOs from linear hydrocarbons. Because of the desired linearity of the feed stock the invention is especially applicable to the conversion of feed stocks produced from Fischer-Tropsch type processes which typically produce linear products such as linear paraffins and linear oxygenates, e.g., linear alcohols, and minor amounts of linear (fatty) acids. The invention is also applicable to other linear hydrocarbon feedstocks such as for example linear hydrocarbons derived from petroleum. As used herein, liquid fuel refers to hydrocarbon fractions boiling with the gasoline range and/or middle distillate range, (e.g., diesel fuel and jet fuel). Thus, for example, the term liquid fuel fraction refers to a gasoline fraction, a diesel fuel fraction, a jet fuel fraction or a fraction including both gasoline and middle distillate. (Middle distillate refers to an oil fraction boiling between about 250° F. to about 700° F., which encompasses jet fuel, kerosene and diesel oil.)

As indicated above, the present process comprises dehydrogenation followed by ethenolysis. The dehydrogenation feed should contain at least about 50 wt. % and preferably at least about 70 wt. % C₁₀ and higher linear paraffins or linear hydrocarbons which will be converted to such linear paraffins under the dehydrogenation conditions; for example linear alcohols. Where the dehydrogenation is conducted in the presence of hydrogen as is typically case, the feedstock may tolerate up to about 50 mol % linear oxygenates which will be roughly equivalent to about 1.2 wt % oxygen for a C₄₀ linear hydrocarbon. Since, the linear paraffins contained in the feedstock typically have many carbon atoms in chains and usually only one or two oxygen atoms, the wt. % of oxygen permitted is typically very much less than the mol % of oxygenate species. Where the feedstock contains a noticeable proportion of oxygen on a weight basis, typically about 0.25 wt. % or more, or olefins or impurities it is preferred to hydrogenate the feed prior to the dehydrogenation step to convert the oxygenates and olefins to linear paraffins and any sulfur containing organic compounds to hydrogen sulfide. This hydrogenation is referred to as hydrotreating. Because of the sensitivity of the ethenolysis catalyst to sulfur poisoning it is preferred to use hydrogenation processes which do not use a sulfided catalyst unless steps are taken to ensure that the sulfided catalyst does not produce sulfur contamination for example by using a sulfur guard bed prior to the dehydrogenation and/or ethenolysis catalyst beds described below. Thus with this caveat, any suitable hydrogenation procedure can be used.

Suitable hydrogenation catalysts which can be used include, for example, noble metal from Group VIIIA according to the 1975, rules of the International Union of Pure and Applied Chemistry, such as platinum or palladium on an alumina or siliceous matrix, or unsulfided Group VIIIA and Group VIB, such as nickel-molybdenum or nickel-tin on an alumina or siliceous matrix. U.S. Pat. No. 3,852,207 granted Mar. 26, 1973, to Stangeland et al., describes a suitable noble metal catalyst and mild conditions, and is herein incorporated by reference. Other suitable, but less desirable, catalysts are detailed, for example, in U.S. Pat. No. 4,157,294, and U.S. Pat. No. 3,904,513 also herein incorporated by reference. The non-noble metal (such as nickel-molybdenum) hydrogenation metal are usually present in the final catalyst composition as oxides. Prior to use, these oxides are converted to sulfides, and to prevent contamination of the other parts of the process with sulfur, it is preferable that steps be taken to avoid contamination (external sulfiding, sulfur guard beds, etc). Preferred non-noble metal overall catalyst compositions contain in excess of about 5 weight percent, preferably about 5 to about 40 weight percent molybdenum and/or tungsten, and at least about 0.5, and generally about 1 to about 15 weight percent of nickel and/or cobalt determined as the corresponding oxides. The noble metal (such as platinum) catalysts contain in excess of 0.01% metal, preferably between 0.1 and 1.0% metal. Combinations of noble metals may also be used, such as mixtures of platinum and palladium. The hydrogenation components can be incorporated into the overall catalyst composition by any one of numerous procedures.

The hydrogenation components can be added to matrix component by co-mulling, impregnation, or ion exchange and the Group VI components, i.e.; molybdenum and tungsten can be combined with the refractory oxide by impregnation, co-mulling or co-precipitation. They are usually added as a metal salt, which can be thermally converted to the corresponding oxide in an oxidizing atmosphere or reduced to the metal with hydrogen or other reducing agent. Suitable matrix materials include synthetic or natural substances as well as inorganic materials such as clay, silica, alumina, amorphous silica-alumina, silica-magnesia, silica-zirconia, silica-thoria, silica-berylia, silica-titania as well as ternary compositions, such as silica-alumina-thoria, silica-alumina-zirconia, silica-alumina-magnesia, and silica-magnesia zirconia. The latter may be either naturally occurring or in the form of gelatinous precipitates or gels including mixtures of silica and metal oxides naturally occurring clays which can be composited with the catalyst include those of the montmorillonite and kaolin families. These clays can be used in the raw state as originally mined or initially subjected to calumniation, acid treatment or chemical modification. A neutral matrix material is preferably used to avoid skeletal isomerization of the hydrocarbon feed and olefin product. Non-zeolitic molecular sieves which can be used include, for example, silicoaluminophosphates (SAPO), ferroaluminophosphate. titanium aluminophosphate and the various ELAPO molecular sieves can be found in U.S. Pat. Nos. 5,114,563 (SAPO); 4,913,799 and the various references cited in U.S. Pat. No. 4,913,799, hereby incorporated by reference in their entirety. Mesoporous molecular sieves can also be included, for example the M41S family of materials (J. Am. Chem. Soc. 1992, 114, 10834-10843), MCM-41 (U.S. Pat. Nos. 5,246,689; 5,198,203; 5,334,368) and MCM-48 (Kresge et al., Nature 359 (1992) 710.)

Preferred hydrotreating conditions will vary with the particular feedstock and hydrotreating catalyst and accordingly can vary over a wide range. Typically hydrotreating is conducted at temperatures varying from about 300° F. (149° C.) to 800° F. (427° C.), pressures in about the range of 50 to 280 atms and liquid hourly space velocity in the range of about from 0.25 to 2 hr⁻¹. Typically, the hydrogen feed is contacted with about from 50 SCF of hydrogen per Bbl of hydrocarbon feed and preferably between about 1,000 to 5,000 SCF/Bbl.

Preferably, the feedstock actually fed to the dehydrogenation reaction should have a high proportion of linear paraffins, at least 50 wt. % more preferably at least about 70 wt. %, and still more preferably at least 90 wt. %, having at least about 10 carbon atoms and up to 400 carbon atoms or more, depending on the molecular size of the NAOs which are desired and the amount of recycle which is acceptable. At the present time there is generally a commercial preference for C₆-C₂₄ NAOs. Where C₆-C₂₄ NAOs are desired the linear paraffinic feedstock will preferably have less than about 200 carbon atoms, more preferably less than about 100 carbon atoms and still more preferably less than about 50 carbon atoms. Also in this case, unless large amounts of lower NAOs are desired, e.g., C₆-C₁₂ NAOs, it is preferred to use feedstocks essentially having at least 16 carbon atoms, more preferably at least 20 carbons and most preferably at least 26 carbon atoms to facilitate the removal of unreacted paraffins and internal olefins on the basis of boiling point. Thus, in general it is preferred that the nominal carbon atom range of the linear hydrocarbon feedstock is higher than the upper end of the desired NAO carbon number range.

The petroleum derived feedstocks may initially containing a substantial proportion, or even a major proportion, of non-linear hydrocarbons as well as the linear hydrocarbons useful in the present process. The desired linear hydrocarbons, e.g., linear paraffins, can be recovered from the petroleum derived feedstocks by any suitable process and can be recovered by a number of known extraction or adsorption processes such as for example, extraction of normal paraffins with urea, adsorption, of normal paraffins on molecular sieves and deoiling. Deoiling refers to an extraction process in which the feedstock is contacted with a solvent, typically a mixture of a ketone (such as methylethylketone) and an aromatic (such as toluene) under temperatures greater than 0° C. The normal paraffins crystallize from the solution, and are separated from the oil.

As is well known the feedstock typically will be a mixture of paraffins having different molecular weights and correspondingly the NAO product from the ethenolysis step will also be a mixture of different molecular size NAOs. The dehydrogenation step can be conducted by passing the linear paraffin feed over a dehydrogenation catalyst under dehydrogenating reaction conditions. As above noted the dehydrogenation is typically conducted in the presence of hydrogen and correspondingly a certain percentage of oxygenates, e.g., linear alcohols, will be hydrogenated to the corresponding paraffins and then dehydrogenated to the corresponding internal olefins. Thus, the linear hydrocarbon feed may contain a substantial amount of linear oxygenates. On a mole percent basis this may be up to about 50 mol. % linear oxygenates though preferably less than 30 mol. %. On a weight percent basis of oxygen this will generally be much less, because the linear hydrocarbons are typically made up of only one or two oxygen atoms per molecule.

In order to reduce or eliminate the amount of diolefins produced or other undesired byproducts the reaction conversion to internal olefins should preferably not exceed about 50% and more preferably should not exceed 30% based on the linear hydrocarbon content of the feed. Preferably the minimum conversion should be at least about 15 wt. % and more preferably at least about 20 wt. %. Because of the low dehydrogenation conversions, a preference is given to feedstocks having a higher proportion of linear hydrocarbons having carbon atom numbers at least in the upper range of the desired NAO products and more preferably greater than the desired NAO fractions to facilitate separation of the desired NAO's based on boiling point differences between the NAO and unreacted paraffins. Preferably the final carbon numbers in the NAO product should range no more than 50 carbon atoms from the initial linear paraffinic hydrocarbon feed more preferably no more than 25 carbon atoms and most preferably no more than 10 carbon atoms in order to reduce the amount of NAOs produced in the first pass which are heavier (longer chained) than the desired NAO product range and correspondingly the need to hydrotreat and recycle the heavier NAOs back to the dehydrogenator.

The dehydrogenation is typically conducted at temperatures in the about the range of from 500 to 1000° F. (260 to 538° C.) preferably about from 600 to 800° F. (316 to 427° C.) at pressures in about the range of 0.1 to 10 atms, more preferably about from 0.5 to 4 atms absolute pressure (about 0.5 to 4 bars) and a LHSV (liquid hourly space velocity) of about from 1 to 50 hr⁻¹ preferably about from 20 to 40 hr⁻¹. Since the longer chained paraffins are more easy to dehydrogenate than the shorter chained paraffins more rigorous conditions, e.g. higher temperatures and/or lower space velocities, within these ranges are typically used where shorter chain paraffins are dehydrogenated and conversely lower temperatures and/or higher space velocities, within these ranges, are typically used where longer chained paraffins are dehydrogenated. The dehydrogenation is also typically conducted in the presence of a gaseous diluent, typically and preferably hydrogen. Although hydrogen is the preferred diluent, other art-recognized diluents may also be used, either individually or in admixture with hydrogen or each other, such as steam, methane, ethane, carbon dioxide, and the like diluents. Hydrogen is preferred because it serves the dual-function of not only lowering the partial pressure of the dehydrogenatable hydrocarbon, but also of suppressing the formation of hydrogen-deficient, carbonaceous deposits on the catalytic composite. Hydrogen is typically utilized in amounts sufficient to ensure a hydrogen to hydrocarbon feed mole ratio of about from 2:1 to 40:1, preferably in the range of about from 5:1 to 20:1.

Suitable dehydrogenation catalysts which can be used include Group VIII noble metals, e.g., platinum, preferably on an oxide support. Less desirably combinations of Group VIII non-noble and Group VIB metals or their oxides; e.g., chromium oxide, may also be used. Suitable catalyst supports include, for example, silica, silicalite, zeolites, molecular sieves, activated carbon alumina, silica-alumina, silica-magnesia, silica-thoria, silica-berylia, silica-titania, silica-aluminum-thora, silica-alumina-zirconia kaolin clays, montmorillonite clays and the like. In general platinum on alumina or silicalite afford very good results in this reaction. Typically, the catalyst contains about from 0.01 to 5 wt. %, preferably 0.1 to 1 wt. % of the dehydrogenation metal (e.g., platinum). Combination metal catalysts such as for example described in U.S. Pat. Nos. 4,013,733; 4,101,593 and 4,148,833 hereby incorporated by reference in their entirety, can also be used.

Preferably, hydrogen and any light gases, such as water vapor formed by the hydrogenation of oxygenates, or hydrogen sulfide formed by the hydrogenation of organic sulfur is removed from the reaction product prior to ethenolysis, for example, by using one or more vapor/liquid separators. In general where the feedstock is hydrotreated prior to the dehydrogenation these gases will be removed by gas/liquid phase separation following the hydrotreatment Since the dehydrogenation produces a net gain in hydrogen, the hydrogen may be taken off for other plant uses or as is typically the case, where the dehydrogenation is conducted in the presence of hydrogen, a portion of the recovered hydrogen can be recycled back to the dehydrogenation reactor. Further information regarding dehydrogenation and dehydrogenation catalysts can, for example, be found in U.S. Pat. Nos. 4,046,715; 4,101,593; and 4,124,649 hereby incorporated by reference in their entirety. A variety of commercial processes also incorporate dehydrogenation processes, in their overall process scheme, which dehydrogenation processes may also be used in the present process to dehydrogen the paraffinic hydrocarbons. Examples of such processes include the dehydrogenation process portion of the Pacol process for manufacturing linear alkylbenzenes, described in Vora et al. Chemistry and Industry, 187-191 (1990); Schulz R. C. et al. Second World Conference on Detergents, Montreaux, Switzerland (October-1986); Vora et al., Second World Surfactants Congress, Paris France (May 1988), and U.S. Pat. No. 5,276,231, hereby incorporated by reference in their entirety.

Preferably diolefins produced during the dehydrogenation are removed prior to ethenolysis sufficiently to reduce the diolefin concentration below about 5 wt. %, more preferably below about 1 wt. %. This can be effected, for example, by suitable adsorption processes or selective hydrogenation processes which selectively hydrogenate diolefins to monoolefins without significantly hydrogenating monoolefins. Suitable, adsorption processes comprise/are described in Chemical Technology of Petroleum, 3^{rd} Edition, by William Gruse and Donald Stevens, pages 310-326 (1960). A variety of adsorbents can be used including aluminas, bauxite, clays and carbon. Aluminas and bauxite are preferred because of their effectiveness and low cost. One selective hydrogenation process known as the DeFine process is described in the Vora et al. Chemistry and Industry publication cited above, hereby incorporated by reference. Suitable selective hydrogenation processes for hydrotreating diolefins to monoolefins without hydrogenating monoolefins are also, for example, described in U.S. Pat. Nos. 4,523,045; 4,523,048 and 5,012,021 hereby incorporated by reference. Typically, the catalyst used for the selective dehydrogenation is either nickel sulfide or palladium in each case dispersed on an inorganic oxide support such as, for example, alumina. Where palladium is used the mole ratio of hydrogen to diolefin typically is in the range of about from 0.25 to 4 moles of hydrogen per mole of diolefin and where nickel sulfide is used about from 1 to 1.8 mole of hydrogen per mole of diolefin. Further details regarding the selective dehydrogenation can be had from the references incorporated by reference hereinabove. Where a sulfide catalyst is used in either the dehydrogenation or the selective hydrogenation it is desirable to pass the monoolefin product through a sulfur adsorption bed prior to the ethenolysis step though this may not be needed in these cases where the monoolefin product from the dehydrogenation or the selective hydrogenation undergo subsequent diolefin removal treatments which also effectively remove sulfur contamination. The preferred method of diolefin removal is by selective hydrogenation because of its efficiency and the fact that it converts the diolefins into monoolefins rather than a waste stream.

Because of the susceptibility of the ethenolysis catalyst to conjugated diene fouling it is preferred to reduce the conjugated diolefin level to below about 50 ppm, more preferably below about 20 ppm, and most preferably below about 5 ppm or lower, to increase the life of the ethenolysis catalyst. This can be done by contacting the dehydrogenation product with a dienophile, preferably maleic anhydride, via a Diels-Alder reaction resulting in the formation of an adduct of the dienophile and diene without significantly affecting the monoolefin. Unless the diolefin concentration of he dehydrogenation product is very low, the concentration of diolefins is preferably reduced by one of the processes for removing diolefins mentioned above before applying the Diels-Alder reaction. Further information regarding Diels-Alder reactions can, for example, be found in Organic Chemistry, 2^{nd} Edition. Morrison and Boyd, Publishers Allyn and Bacon, Inc. Boston, October 1969, page 975-976 and Dienes in the Diels Alder Reaction, Fringuelli and Taticchi, Publishers Wiley-Interscience New York (1990) pp. 4-19, hereby incorporated by reference, and the references cited therein. The Diels-Alder separation step can, for example, be conducted by contacting the dehydrogenation reaction product with a suitable dienophile, preferably maleic anhydride, under reactive conditions at temperatures in the range of about from 20 to 250° C. preferably about from 100 to 150° C. The dienophile preferably should be one which is itself easily separated from the monoolefin product and which yields an adduct which is also easily separated from the monoolefin product. Suitable dieneophiles which can be used, include, for example, maleic anhydride, methyl maleic anhydride, ethyl maleic anhydride, acrylonitrile, methacrylonitrile, 1,4 benzoquinone, methyl-1,4 benzoquinone and the like and compatible mixtures thereof. Since, maleic anhydride is generally the dieneophile of choice for this separation, both because of its performance and also because it is relatively inexpensive, the following discussion has been directed to maleic anhydride, however, it should be appreciated that other dienophiles having similar properties (e.g., ease of separation of both the dienophile and the adduct) could also be used.

The Diels-Alder reaction may be advantageously conducted as a two-phase liquid:liquid reaction or solid:liquid reaction using maleic anhydrate in molten form or dissolved in an inert immiscible organic solvent or dispersed on an inorganic solid support. The maleic adduct is formed in the maleic anhydride phase and thus may be removed by decantation of the molten maleic anhydrate phase or the immiscible organic solvent phase. Suitable immiscible inert organic solvents which can be used should not react with the ionic liquid, and should not have great solubility with the paraffins. While toluene and other aromatics are typically used as solvents for maleic anhydride in the Diels-Alder reaction, these solvents are less desireable here because they tend to be too miscible with the paraffin. Thus preferred solvents include for example ionic liquids, tetrahydrofuran, dioxane, glymes and the like and compatible mixtures thereof. Where the maleic anhydride is dispersed on an inorganic support, for example in a fixed bed reactor, the dehydrogenation product is simply passed through the supported solid maleic anhydride with the adduct forming and separating out on the support. When the maleic anhydride is spend it can be discharged from the reactor and replaced with a new charge of maleic anhydride dispersed on the support. Suitable inert organic supports which can be used include, for example, carbon, silica, keiselgur, alumina and the like and mixtures thereof. Following removal of the maleic anhydride-diene adduct the olefin product stream is preferably scrubbed with water or caustic, e.g., dilute aqueous sodium hydroxide, to remove any traces of maleic anhydride and adduct. Following scrubbing, water is removed from the monoolefin product for example, by stripping or adsorption, to reduce the water content to below about 500 ppm, preferably below about 100 ppm and more preferably below about 50 ppm, since water can also poison the ethenolysis. Further details regarding the removal of conjugated dienes from monoolefins can be had by reference to Provisional Application Serial No. ______, of Saleh Elomari, Norman Reynolds and Steven Herron entitled Process For The Removal Of Conjugated Olefins From A Monoolefin Stream filed on even date herewith, assigned to Chevron Chemical Company LLC and hereby incorporated by reference in its entirety.

If desired, branched hydrocarbons may be removed before or after the dehydrogenation process or after the ethenolysis process described below by any suitable process, typically by adsorption. One commercial adsorption process for removing branched hydrocarbons and aromatics from linear paraffins is known as the Molex or Sorbex process described in McPhee, Petroleum Technology Quarterly, pages 127-131, (Winter 1999/2000) which description is hereby incorporated by reference.

The internal olefin reaction product, or the internal olefin portion thereof, preferably purified to remove diolefins as noted above is contacted with ethylene in the presence of an ethenolysis catalyst under ethenolysis reaction conditions resulting in the production of NAOs having smaller chain lengths than the corresponding internal olefins. As is well known, the disproprontionation of the internal olefin with ethylene (i.e., ethenolysis) results in the production of two shorter chained lengthen NAOs; the relative chain length of which depends on the position of the double bond, for example,

CH₃(CH₂)₁₀CH═CH(CH₂)₁₀CH₃+CH₂═CH₂→2CH₃(CH₂)₁₀CH═CH₂

CH₃(CH₂)₁₂CH═CH(CH₂)₆CH₃ +CH₂═CH₂→CH₃(CH₂)₁₂CH═CH_{2+CH}₃(CH₂)₆CH═CH₂

Although in theory any ethenolysis or olefin disportionation catalyst can be used for the reaction, it is preferred to use rhenium or tungsten as the catalyst, preferably supported on one of the classes of supports described above, and more preferably a neutral support such as silica, alumina, or titania. The reaction is typically conducted at temperatures in about the range of 50° F. to 600° F. (10° C. to 315° C.) preferably about from 70° F. to 500° F. (21° C.-260° C.), pressures of about from 1 to 15 atms (1.01- 15.2 bars), preferably about from 2 to 12 atms using an ethylene to internal olefin mole ratio of 5 to 20 moles of ethylene per mole of internal olefin preferably about from 8 to 15 moles of ethylene per mole of olefin, and a LHSV space velocity of about from 0.1 to 10 hr⁻¹ preferably about from 0.2 to 2 hr⁻¹. Optimum temperature and pressure ranges typically will vary with the particular catalyst used. Thus, where ruthenium is used as the catalyst it is preferred to use temperatures in the range of about from 60 to 80° F. (16 to 27° C.) preferably about from 65 to 75° F. (18 to 24° C.) and pressures in the range of about 1.5 to 3 atms and where tungsten is used as the catalyst to use reaction temperature in the range of about from 400° F. to 600° F. (204 to 316° C.), preferably about from 450° F. to 550° F. (232 to 288° C.) and pressures in the range of about from 8 to 12 atms preferably about 9 to 11 atms. Typically high conversions of internal olefins are obtained, typically greater than 50% and often on the order of 85-95%, with essentially 100% selectivity of the reacted linear olefins to NAOs. Since ethenolysis is adversely affected by the presence of sulfur, typically found in the form of organic sulfur compounds, it is important that the sulfur content of the ethenolysis feed is low, preferably less than 1 ppm sulfur, and more preferably less than 0.1 ppm of sulfur. We have found that, in general, paraffin products produced by Fischer-Tropsch type processes have very low sulfur contents as well as high linearity and thus ideal feedstocks for ethenolysis after conversion to internal olefins. As already mentioned above, such sulfur compounds can be removed prior to the ethenolysis reaction by hydrotreating the feedstock prior to the dehydrogenation reaction.

Unreacted ethylene is separated from the reaction product and may be purified to remove hydrogen and any NAO products boiling between ethylene and the desired product fraction and then recycled back to the ethenolysis reactor or reactor section. Concurrently with removal of the ethylene or after removal, the NAO reaction product can be separated to recover the desired chain length NAO product fractions. Longer chain length NAOs can be recycled or subjected to other processing to prepare products such as fuels or lubricating oils. As above mentioned, in general it is preferred to recover a NAO product having a chain length lower than the nominal chain length range of the linear paraffin feed to the dehydrogenation reaction. This will facilitate the separation of the unreacted paraffins and internal olefins in the ethenolysis reaction product by relatively conventional separation procedures based on boiling points differences. For example, in the case of a C₄₀-C₄₀₀ linear paraffin feedstock, the C₄₀ and higher NAO products can be recycled and the C₃₉ and lower NAO recovered essentially free of unreacted paraffins and internal olefins. Also, if desired selective recycling of differing NAO fractions may be effected for example where a different molecular weight linear feedstock is being used from what was originally used. Where lower molecular weight linear paraffin feed streams are used, for example C₁₀-C₅₀, and it is desired to recover NAOs overlapping the lower end of the paraffin feed stream, for example C₆-C₂₄, it may be necessary to employ more sophisticated procedures such as, for example, extractive distillation and/or adsorption, to remove the unreacted paraffins and internal olefins boiling in about the same range as the desired NAO fraction. By employing the dehydrogenation controls and molecular weight feedstock and NAO product selection set forth above NAO fractions, for example C₆-C₂₄ NAO, having a NAO purity of at least 70 wt. % and more typically a purity of at least 90 wt. % preferably at least 95 wt. %, can be obtained using conventional separation techniques such as for example fractional distillation. By using more sophisticated separation techniques, such as extractive distillation or involving absorption, NAO purities of at least 98 wt. % and approaching 100% can be obtained.

The dehydrogenation and ethenolysis reactions may be conducted in a variety of reactors for example, fixed bed, ebulated bed, fluidized bed. In general it is preferred to conduct the reactions in one or more fixed catalyst bed reactors containing one or more catalyst beds. Less desirability, because of the production of diolefin in the dehydrogenation, both reactions may in some case also be conducted in a single reactor by using a multi-bed reactor; however in general it is much preferred to conduct each reaction separately with intermediate removal of diolefins. Distillation reactors can also be used for the ethenolysis reaction. By using a distillation reactor the NAO reaction product and unreacted ethylene can be removed from the reactor as separate product streams. The ethylene stream may then be recycled back to the distillation reactor whereas the NAO product fraction can be fractionated into the desired NAO product fractions ranges and higher boiling NAOs, and unreacted internal olefins and linear paraffins.

In a further embodiment the invention provides an integrated ethenolysis and hydrocracking process for upgrading linear hydrocarbons into more valuable NAOs and liquid fuel and/or lube oil base stocks. In this case, at least a portion of the higher boiling linear hydrocarbons, e.g., boiling in the C₅₀ and higher paraffin range and/or the higher boiling fraction of the ethenolysis reaction, product, including NAO boiling above the desired NAO product fraction or a portion thereof, is hydrocracked to hydrocarbons boiling in the liquid fuel range (about from 68° F. to 700° F.; 20° to 371° C.). This may be effected by hydrocracking the respective fractions separately or by combining one or more of the fractions prior to hydrocracker. Preferably, fractions having similar boiling point ranges, are combined. The hydrocracking operation can be conducted as a block operation wherein the hydrocracker is alternated between gasoline fuel fractions and heavier fuel fractions or lube oil fractions or parallel hydrocrackers can be used wherein each hydrocracker train processes a different distillation range feedstock. Hydrocracking can be effected by contacting the particular fraction or combination of fractions, with hydrogen in the presence of a suitable hydrocracking catalyst at temperatures in the range of about from 600 to 900° F. (316 to 482° C.) preferably 650 to 850° F. (343 to 454° C.) and pressures in the range about from 200 to 4000 psia (13-272 atm preferably 500 to 3000 psia (34-204 atm) using space velocities based on the hydrocarbon feedstock of about 0.1 to 10 hr-1 preferably 0.25 to 5 hr-1. Generally, more severe conditions within these ranges will be used with higher boiling feedstocks and depending on whether gasoline, middle distillate or lubricating oil is desired as the primary economic product. The hydrocracking step reduces the size of the hydrocarbon molecules, hydrogenates olefin bonds, hydrogenates aromatics, and removes traces of heteroatoms resulting in an improvement in fuel or base oil product quality.

As is well known the hydrocracking catalysts contain a hydrogenation component and a cracking component. The hydrogenation component is typically a metal or combination of metals selected from Group VIII noble and non-noble metals and Group VIB metals. The noble metals, particularly platinum or palladium, are generally more active but are expensive. Non-noble metals which can be used include molybdenum, tungsten, nickel, cobalt, etc. Where non-noble metals are used it is generally preferred to use a combination of metals, typically at least one Group VIII metal and one Group VIB metal, e.g., nickel-molybdenum, cobalt-molybdenum, nickel-tungsten, and cobalt-tungsten. The non-noble metal hydrogenation metal are usually present in the final catalyst composition as oxides, or more preferably, as sulfides when such compounds are readily formed from the particular metal involved. Preferred non-noble metal overall catalyst compositions contain in excess of about 5 weight percent, preferably about 5 to about 40 weight percent molybdenum and/or tungsten, and at least about 0.5, and generally about 1 to about 15 weight percent of nickel and/or cobalt determined as the corresponding oxides. The sulfide form of these metals is most preferred due to higher activity, selectivity and activity retention.

The hydrogenation components can be incorporated into the overall catalyst composition by any one of numerous procedures. They can be added either to the cracking component or the support or a combination of both. In the alternative, the Group VIII components can be added to the cracking component or matrix component by co-mulling, impregnation, or ion exchange and the Group VI components, i.e. molybdenum and tungsten can be combined with the refractory oxide by impregnation, co-mulling or co-precipitation. Although these components can be combined with the catalyst support as the sulfides, that is generally not the case. They are usually added as a metal salt which can be thermally converted to the corresponding oxide in an oxidizing atmosphere or reduced to the metal with hydrogen or other reducing agent. The non-noble metal composition can then be sulfided by reaction with a sulfur donor such as carbon bisulfide, hydrogen sulfide, hydrocarbon thiols, elemental sulfur, and the like.

The cracking component is an acid catalyst material and may be a material such as amorphous silica-alumina or may be a zeolitic or non-zeolitic crystalline molecular sieve. Examples of suitable hydrocracking molecular sieves include zeolite Y, zeolite X and the so called ultra stable zeolite Y and high structural silica:alumina ratio zeolite Y such as for example described in U.S. Pat. Nos. 4,401,556, 4,820,402 and 5,059,567. Small crystal size zeolite Y, such as described in U.S. Pat. No. 5,073,530 can also be used. The disclosures of all of which patents are hereby incorporated by reference in their entirety. Non-zeolitic molecular sieves which can be used include, for example silicoaluminophosphates (SAPO), ferroaluminophosphate, titanium aluminophosphate and the various ELAPO molecular sieves described in U.S. Pat. No. 4,913,799 and the references cited therein. Details regarding the preparation of various non-zeolite molecular sieves can be found in U.S. Pat. Nos. 5,114,563 (SAPO); 4,913,799 and the various references cited in U.S. Pat. No. 4,913,799, hereby incorporated by reference in their entirety. Mesoporous molecular sieves can also be included, for example the M41S family of materials (J. Am. Chem. Soc. 1992, 114, 10834-10843), MCM-41 (U.S. Pat. Nos. 5,246,689, 5,198,203, 5,334,368), and MCM-48 (Kresge et al., Nature 359 (1992) 710.)

In general amorphous silica-alumina is more selective for middle distillates, e.g., diesel fuel, whereas crystalline molecular sieves are much more active and produce greater amounts of lighter products, e.g., gasoline. The so-called high (structural) silica-alumina ratio (Si2O3:Al2O3≧about 50) Y zeolites are less active than the conventional zeolite Y but, are more selective for middle distillate and more active than amorphous silica-alumina. The catalyst also typically contains a matrix or binder material resistant to the conditions used in the hydrocracking reaction. Suitable matrix materials include synthetic or natural substances as well as inorganic materials such as clay, silica and/or metal oxides. The latter may be either naturally occurring or in the form of gelatinous precipitates or gels including mixtures of silica and metal oxides naturally occurring clays which can be composited with the catalyst include those of the montmorillonite and kaolin families. These clays can be used in the raw state as originally mined or initially subjected to calumniation, acid treatment or chemical modification.

The catalyst may be composited with a porous matrix material, such as alumina, silica-alumina, silica-magnesia, silica-zirconia, silica-thoria, silica-berylia, silica-titania as well as ternary compositions, such as silica-alumina-thoria, silica-alumina-zirconia, silica-alumina-magnesia, and silica-magnesia zirconia. The relative proportions of molecular sieve component and inorganic oxide matrix or binder may vary widely with the molecular sieve content ranging from between 1 to 99, more usually 5 to 80, percent by weight of the composite. The matrix may itself possess catalytic properties generally of an acidic nature, such as for example where amorphous silica-alumina is used as a matrix or binder for a molecular sieve. In general we prefer to use a non-zeolite or low acidic zeolite catalyst, e.g., high structural silica:alumina ratio Y zeolite, as the catalyst where middle distillates is desired as the main commercial product and an acidic zeolite catalyst, e.g., conventional or ultra stabilized Y zeolite, where gasoline is desired as the main commercial product.

Furthermore more than one catalyst type may be used in the reactor. The different catalyst types can be separated into layers or mixed.

The hydrocrackate is then separated into various boiling range fractions. The separation is typically conducted by fractional distillation preceded by one or more vapor-liquid separators to remove hydrogen and/or other tail gases. The fractions separated will typically include a gasoline fraction and a high boiling bottom fraction and one or more intermediate boiling range fractions. The high boiling fraction is preferably recycled back to the hydrocracker. The light tail gas fraction, i.e., methane, ethane, proposal and any residual hydrogen is withdrawn and can be for fuel gases or for hydrogen recovery which in turn can be recycled back to the hydrocracker. Typical, liquid/vapor separator systems which can be used to remove tail gases and hydrogen are, for example, described in U.S. Pat. Nos. 3,402,122 and 4,159,937 hereby incorporated by reference in their entirety.

It is also preferred to hydrotreat the feed prior to hydrocracking by any suitable hydrotreating process to remove oxygenates and sulfur compounds. Hydrotreating is well known to the art and has already been discussed in detail above with respect to hydrotreating the dehydrogenation feed. Typically, hydrotreating of the hydrocracker feed is conducted at temperatures in about the range of 650° F. to 800° F.(343° C.-427° C.) and pressures in about the range of 800 to 3000 psi (54 to 204 atms) in the presence of a catalyst comprising at least one Group VIII or Group VI metal and more typically containing one metal from each group, e.g., cobalt-molybdenum; nickel-tungsten, on a neutral mineral oxide support such as alumina and the like. Hydrotreating may be conducted in a separate reactor preceding the hydrocracking or may be conducted in the same reactor, for example, as one or more hydrotreating catalyst beds preceding one or more hydrocracking catalyst beds. The hydrotreating bed may also serve as a screen to remove any particulate matter in the feedstock, for example catalyst fines from a previous reaction, or may itself be preceded with guard beds of crushed rock or other suitable material.

In another embodiment of the invention, C₁-C₃ alkanes gases, e.g., natural gas, are reformed to a mixture of hydrogen and carbon monoxide, e.g., syngas. Starting with the C₁-C₃ alkanes gases the alkanes are reformed to a mixture of hydrogen and carbon monoxide. Reforming is well known in the art, and includes a variety of technologies including steam reforming, partial oxidation, dry reforming, series reforming, convective reforming, and autothermal reforming. All have in common the production of syngas from methane and other light hydrocarbons, and an oxidant (steam, oxygen, carbon dioxide, air, enriched air or combinations). The effluent typically contains some carbon dioxide and steam in addition to syngas and unreacted feed gases. Series reforming, convective reforming and autothermal reforming incorporate exothermic and endotheric syngas forming reactions in order to better utilize the heat generated in the process. These processes for producing synthesis gas or syngas from C₁-C₃ alkanes are well known to the art.

Steam reforming is typically effected by contacting C₁-C₃ alkanes with steam, preferably in the presence of a reforming catalyst at a temperature in the range of about 1300° F. (705° C.) to about 1675° F. (913° C.) and pressures from about 10 psia (0.7 bars) to about 500 psia (34 bars). Suitable reforming catalysts which can be used include, for example, nickel, palladium, nickel-palladium alloys, and the like. Additional information regarding steam reforming C₁-C₃ alkanes, e.g., methane, to syngas can be found in U.S. Pat. No. 5,324,335 hereby incorporated by reference in its entirety.

Partial oxidation of C₁-C₃ alkanes to syngas is also conducted at high temperature and while the partial oxidation may be conducted without a catalyst it is more effectively conducted in the presence of a catalyst. In general Group VIII metals can be used as the catalyst typically supported on a mineral oxide or synthetic support, e.g., alumina. Typically, the partial oxidation is conducted at temperatures in about the range of 1500° F. (815° C.) to about 2000° F. (1093° C.) pressures in about the range from atmospheric to 3000 psia (1 to 20.4 bars). Space velocities can vary over a very wide range and typical range of 100 to 100,000 hr⁻¹ and even higher depending on the particular catalyst used and the type of reactor. A discussion of nickel silica alumina and nickel/magnesium oxide and cobalt/magnesium oxide and other oxidation catalysts may be found in A. Santos et al., Oxidation of Methane to Synthesis Gas in Fluidized Bed Reactor using MgO-Based Catalysts, Journal of Catalysis, Vol. 158 (1996) pp. 81-91 hereby incorporated by reference in its entirety.

The partial oxidation may also be conducted using a peroskite catalyst partial oxidation process such as described in U.S. Pat. No. 5,149,516 hereby incorporated by reference in its entirety. Peroskites are materials having essentially the same crystal structure as the mineral peroskite (Ca Ti O3) without limitation as to the elemental constituents thereof. Such materials can be represented by the formula XYO3 wherein X and Y can be variety of elements. For example, X can be La, Ca, Sr, Ba, Na, K, Ag, Cd and mixtures thereof and Y can be Ta, Co, Ti, Ga, Nb, Fe, Ni, Mn, Gr, V, Th, Pb, Sn, Mo, Zn and mixtures thereof. Partial oxidation reactions using a peroskite catalyst are typically conducted at temperatures in the range of about from 600 to 900° C., pressures of about from 0.1 to 100 bar and gas hourly space velocities of from 100 to 300,000 hr⁻¹. (These space velocities are determined using a gas volume based on NTP conditions, i.e. room temperature (about 25° C.) and one atmosphere of pressure.) The mole ratio of lower alkane can vary from 1:1 to 100:1 moles of alkane to oxygen. Regardless of the system used to produce syngas it is desirable to remove any sulfur compounds, e.g., hydrogen sulfide and mercaptans, contained in the C₁-C₃ alkane feed. This can be effected by passing the C₁-C₃ alkanes gas through a packed bed sulfur scrubber containing zinc oxide bed or another slightly basic packing material. If the amount of C₁-C₃ alkanes exceeds the capacity of the synthesis gas unit the surplus C₁-C₃ alkanes can be used to provide energy throughout the facility. For example, excess C₁-C₃ alkanes may be burned in a steam boiler to provide the steam used in the thermal cracking step of the present process.

The syngas product is converted to liquid hydrocarbons by contact with a Fischer-Tropsch catalyst under reactive conditions. Depending on the quality of the syngas it may be desirable to purify the syngas prior to the Fischer-Tropsch reactor to remove carbon dioxide produced during the syngas reaction and any sulfur compounds, if they have not already been removed. This can be accomplished by contacting the syngas with a mildly alkaline solution (e.g., aqueous potassium carbonate) in a packed column. In general Fischer-Tropsch catalysts contain a Group VIII transition metal on a metal oxide support. The catalyst may also contain a noble metal promoter(s) and/or crystalline molecular sieves. Pragmatically, the two transition metals which are most commonly used in commercial Fischer-Tropsch processes are cobalt or iron. Ruthenium is also an effective Fischer-Tropsch catalyst but is more expensive than cobalt or iron. Where a noble metal is used, platinum and palladium are generally preferred. Suitable metal oxide supports or matrices which can be used include alumina, titania, silica, magnesium oxide, silica-alumina, and the like, and mixtures thereof.

Although Fischer-Tropsch processes produce a hydrocarbon product having a wide range of molecular sizes, the selectivity of the process toward a given molecular size range as the primary product can be controlled to some extent by the particular catalyst used. In the present process, it is preferred to produce C₂₀-C₅₀ paraffins as the primary product, and therefore, it is preferred to use a cobalt catalyst although iron catalysts may also be used. One suitable catalyst which can be used is described in U.S. Pat. No. 4,579,986 as satisfying the relationship:

(3+4R)>L/S>(0.3+0.4R),

wherein:

L=the total quantity of cobalt present on the catalyst, expressed as mg Co/ml catalyst

S=the surface area of the catalyst, expressed as m²/ml catalyst, and

R=the weight ratio of the quantity of cobalt deposited on the catalyst by kneading to the total quantity of cobalt present on the catalyst.

Preferably, the catalyst contains about 3-60 ppw cobalt, 0.1-100 ppw of at least one of zirconium, titanium or chromium per 100 ppw of silica, alumina, or silica-alumina and mixtures thereof. Typically, the synthesis gas will contain hydrogen, carbon monoxide and carbon dioxide in a relative mole ratio of about from 0.25 to 2 moles of carbon monoxide and 0.01 to 0.05 moles of carbon dioxide per mole of hydrogen. In the present process we prefer to use a mole ratio of carbon monoxide to hydrogen of about 0.4 to 1, more preferably 0.5 to 0.7 moles of carbon monoxide per mole of hydrogen with only minimal amounts of carbon dioxide; preferably less than 0.5 mole percent carbon dioxide.

In the present process the Fischer-Tropsch reaction is typically conducted at temperatures of about from 300 to 700° F. (149 to 371° C.) preferably about from 400 to 550° F. (204 to 228° C.); pressures of about from 10 to 500 psia, (0.7 to 34 bars) preferably 30 to 300 psia, (2 to 21 bars) and catalyst space velocities of about from 100 to 10,000 cc/g/hr., preferably 300 to 3,000 cc/g/hr. The reaction can be conducted in a variety of reactors for example, fixed bed reactors containing one or more catalyst beds, slurry reactors, fluidized bed reactors, or a combination of different type reactors. The Fischer-Tropsch reaction product can be separated into the desired product fractions, e.g., a gasoline fraction (B.P. about 68-450° F./20-232° C.) a middle distillate fraction (B.P. about 450-650° F./232-343° C.) a wax fraction (B.P. about 650-1100° F./539° C.) primarily containing C₂₀ to C₅₀ normal paraffins with a small amount of branched paraffins and a heavy fraction (B.P. above about 1100° F.) and tail gases. With the exception of the wax fraction, the other fractions are largely a matter of choice depending on the products desired; for example, a single liquid fuel fraction may be taken off comprising both gasoline and middle distillate may be taken off or multiple fuel cuts as well as heavy cuts may be taken. In some cases, for example, where a bubble slurry reactor is used, both liquid and gaseous product streams may be taken off. The gaseous stream will contain tail gases and may also contain gasoline fuel fraction. The gasoline fraction can be recovered using vapor/liquid separators. The tail gas primarily containing hydrogen and C₁ to C₄ paraffins can be used as fuel gas or can be treated to remove carbon dioxide and used as a hydrogen or alkane recycle stream.

In a preferred embodiment, the Fischer-Tropsch reaction is conducted in a bubble column slurry reactor. In this type of reactor synthesis gas is bubbled through a slurry comprising catalyst particles in a suspending liquid. Typically the catalyst has a particle size of about from 10-110 microns, preferably about from 20-80 microns, more preferably about from 25-65 micron and a density of about from 0.25 to 0.9 g/cc preferably about from 0.3-0.75 g/cc. The catalyst typically comprises one of the aforementioned catalytic metals, preferably cobalt on one of the aforementioned catalyst supports. Preferably the catalyst comprises about 10 to 14 wt. % cobalt on a low density fluid support, for example alumina, silica and the like having a density within the ranges set forth above for the catalyst. Since, the catalyst metal may be present in the catalyst as oxides the catalyst is typically reduced with hydrogen prior to contact with the slurry liquid. The starting slurry liquid is typically a heavy hydrocarbon having a viscosity high enough to keep the catalyst particles suspended, typically a viscosity between 4-100 centistokes at 100° C., and a low enough volatility to avoid vaporization during operation, typically an initial boiling point range of about from 350 to 550° C. The slurry liquid is preferably essentially free of contaminants such as sulfur, phosphorous or chlorine compounds. Thus initially, it may be desirable to use a synthetic hydrocarbon fluid such as a synthetic olefin oligomer as the slurry fluid. Ultimately, a paraffin fraction of the product having the desired viscosity and volatility is typically recycled as the slurry liquid. The slurry typically has a catalyst concentration of about 2-40 wt. % catalyst, preferably 5-20 wt. % and more preferably 7-15 wt. % catalyst based on the total weight of the catalyst, i.e., metal plus support. The syngas feed typically has hydrogen to carbon monoxide mole ratio of about from 0.5 to 4 moles of hydrogen per mole of carbon monoxide preferably about from 1 to 2.5 and more preferably about 1.5 to 2.

The bubble slurry reactor is typically operated at temperatures within the range of 150-300° C., preferably 185 to 265° C. and more preferably 210-230° C. and pressures within the range of 1 to 70 bar, preferably 6-35 bar and most preferably 10 to 30 bar (1 bar=14.5 psia). Typical synthesis gas linear velocity ranges in the reactor from about 2 to 40 cm per sec. preferably 6 to 10 cm per sec. Additional details regarding bubble column slurry reactors can, for example, be found in Y. T. Shah et al., Design Parameters Estimations for Bubble Column Reactors, AlChE Journal, 28 No. 3, pp. 353-379 (May 1982); Ramachandran et al., Bubble Column Slurry Reactor, Three-Phase Catalytic Reactors Chapter 10, pp. 308-332 Gordon and Broch Science Publishers (1983); Deckwer et al., Modeling the Fischer-Tropsch Synthesis in the Slurry Phase, Ind. Eng. Chem. Process Des. Dev. v 21, No. 2, pp. 231-241 (1982); Kölbel et al., The Fischer-Tropsch Synthesis in the Liquid Phase, Catal. Rev.-Sci. Eng., v. 21(n), pp. 225-274 (1980) and U.S. Pat. No. 5,348,982, all of which are hereby incorporated by reference in their entirety.

The reaction conditions can be adjusted to produce a gaseous reaction product having a boiling point at about the desired paraffin cut desired in the liquid product such that paraffins and other hydrocarbons having boiling points below the desired paraffin boiling point are taken off in the gaseous reaction product. Thus, the gaseous reaction product from the Fischer-Tropsch bubble slurry reactor will contain hydrocarbons boiling below a selected value in the range of about from 350° F. (C₁₀ a paraffin) to 800° F. (C₂₈ n-paraffin) (e.g., tail gases through middle distillate). The liquid reaction product is recovered as or with the slurry and comprises hydrocarbons boiling above about the selected value, e.g., vacuum gas oil through heavy paraffins. The gaseous reaction product can be separated into a tail gas fraction and a condensate fraction, i.e., about C₅ to the selected value using a high pressure and/or lower temperature vapor-liquid separator or low pressure separators or a combination of separators. The tail gas fraction may be used as described above. The condensate fraction can be fractionated into the desired product fraction; e.g., gasoline, light middle distillate or more preferably is upgraded by hydrocracking. The liquid F-T product after removal of the particulate catalyst, is typically separated into a wax fraction boiling in the range of about the selected value, to 110020 F., for example, primarily about containing C₁₆ to C₅₀ linear paraffins, (where C₆-C₂₄ NAOs are desired with minimum recycle), with relatively small amounts of higher boiling branched paraffins, and depending on the selected value one or more liquid fuel fractions and one or more fractions boiling above about 1100° F. (If higher boiling NAOs are desired or if recycle through the hydrotreater and dehydrogenation is used, higher boiling fractions may be used.) Typically, the separation is effected by fractional distillation. A portion of the liquid reaction product is preferably recycled to provide slurry liquid. The separated wax fraction should contain at least 70 wt. % and preferably at least 90 wt % C₁₆ to C₅₀ linear paraffins, where C₆-C₂₄ NAOs are desired.

Alternatively, if the Fischer-Tropsch reaction is designed to produce a single process stream, for example, by using fixed bed reactor, then the entire product stream may be fractionated generally after first removing hydrogen and preferably other tail gases as well. This can be done by passing the product stream through one or more vapor-liquid separators prior to fractionation.

The selected paraffin fraction is dehydrogenated to the corresponding the internal olefins which are in turn ethenolysized to yield smaller chain length NAO, for example C₆ to C₂₄ NAOs. Dehydrogenation and ethenolysis can be effected by the procedures already described above including hydrotreating and recycling long chain NAOs. Similarly, one or more of the liquid fuel fraction and high boiling fraction (boiling about 1100° F.) of the Fischer-Tropsch product and/or about the C₃₀+ fraction of the ethenolysis reaction product may be upgraded by hydrocracking as already described above, to yield higher quality fuels and lube base oils.

Although in one aspect the invention is described herein in terms of a Fischer-Tropsch reaction product or a Fischer-Tropsch process this aspect of the invention also applies to products prepared the various modifications of the literal Fischer-Tropsch process by which hydrogen (or water) and carbon monoxide (or carbon dioxide) are converted to linear hydrocarbons (e.g., paraffins, alcohols ethers etc.) and to the products of such processes. Thus the term Fischer-Tropsch type product or process is intended to apply to Fischer-Tropsch processes and products and the various modifications thereof and the products thereof. For example, the term is intended to apply to the Kolbel-Engelhardt process typically described by the reactions:

3CO+H₂O→—CH₂—+2CO₂

CO₂+3H₂→—CH₂—+2H₂O

Where products other than normal alpha olefins are obtained it may be desirable to increase the quality of these products for fuels and lubricating oil uses. It is frequently desireable to hydrotreat and/or hydroisomerize hydrocarbons for such uses and to hydrocrack fractions boiling above about 700° F., particularly high boiling fractions, to convert them into more desirable lower boiling fuel and/or lubricating oil fractions. Hydrotreating and hydrocracking have already been discussed above. Hydroisomerization is a dewaxing process which cracks and isomerizes n-paraffins into non-waxy isoparaffins thus reducing pour point and viscosity. Hydroisomerization can be conducted using a variety of intermediate pore size molecular sieve catalysts typically using reaction temperatures in the range of about from 600 to 800° F., pressure of about from 200 to 2,000 psig and liquid space velocities of about ½ to 2 hr⁻¹. Further details regarding such processes can be had by reference to U.S. Pat. Nos. 4,859,311; 5,246,566; and 5,282,958, hereby incorporated by reference in their entirety.

For the purposes of further understanding of the invention specific two non-limiting embodiments of the invention will now be described with reference to the drawing.

### EXAMPLE 1

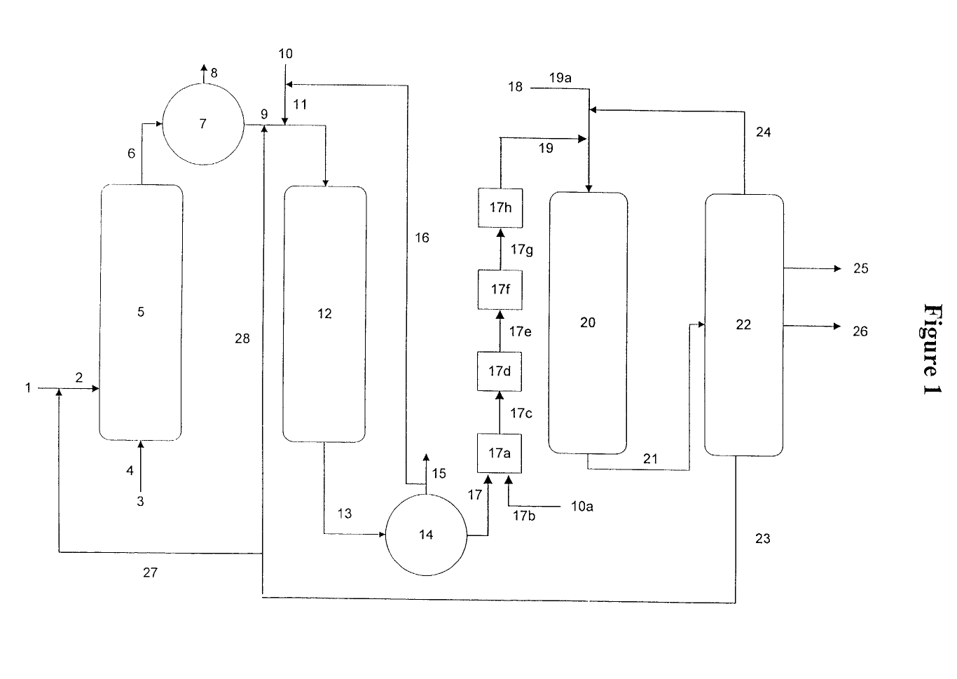

An embodiment of the invention for upgrading a petroleum derived C₃₀-C₂₀₀ linear hydrocarbon feedstock comprising at least 70 wt. % linear paraffins with up to 50 mole % of oxygenates (e.g., linear alcohols) to C₆-C₂₄ NAOs will now be described with reference to FIG. 1. The linear hydrocarbon feed (1) is fed via line 2 to hydrotreater (5) containing a packed bed of platinum on alumina catalyst. Hydrogen (3) is fed to the hydrotreater via line 4 at a ratio of about 3,000 SCF per Bbl of linear hydrocarbon feed. The hydrotreater is operated at a temperature of about 650° F. to 700° F. (343° C. to 371° C.), a pressure of about 10 atm to 20 atm and a liquid space velocity (LHSV) of about 0.5 hr⁻¹ to 1 hr⁻¹. The hydrotreater hydrogenates olefins and oxygenates (e.g., alcohols) in the feed to the corresponding paraffins and converts organics sulfur and nitrogen compounds to hydrogen sulfide and ammonia which are preferably removed from the liquid reaction products as gases along with hydrogen and scrubbed out of the hydrogen gas. The entire hydrogenated product is fed via line 6 to vapor/liquid separator 7 where the gas phase (hydrogen, ammonia, hydrogen sulfide, and any light hydrocarbons e.g., C₁-C₄ alkanes) is separated and discharged via line 8 and the hydrogenated C₃₀-C₂₀₀ hydrocarbon liquid phase fed via line 9 to dehydrogenation reaction 12 along with recycle hydrogen furnished by lines 16-11 and if needed any made-up hydrogen (10) furnished by line 11. Hydrogen is supplied to reactor 12 at a ratio of about 20 moles of hydrogen per mole of hydrocarbon feed, including any recycle. The dehydrogenation reactor is a fixed bed reactor containing 0.5 wt. % platinum on alumina catalyst bed. The reactor is initially set for operation at a LHSV of about 40 hr⁻¹, a temperature of about from 700 F. to 750 F. (371 C. to 399 C.) and at about 2 atm absolute pressure which conditions are then adjusted as needed to give about a 30% conversion of paraffin, to internal olefins; for example, higher LHSVs and lower temperatures give lower conversions and vice versa. The entire reaction product is fed via line 13 to vapor/liquid separator 14 where the hydrogen is taken off via line 15. A portion of the hydrogen is recycled back to reactor 12 via line 16 and the remainder used for other plant purposes.

The liquid reaction product, from separator 14, is fed via line 17 to selective hydrogenator 17a, containing a nickel sulfide on alumina catalyst, operated at a temperature in the range of about 392 to 437° F. (200° C. to 225° C.) a pressure of about 300 to 350 k Pag and a space velocity of about 20 hr⁻¹. Hydrogen (1Oa) is fed to hydrogenator 17a via line 17b at a ratio of about 1.2 to 1.5 moles of hydrogen per mole of diene in the feed furnished via line 17. After removal of excess hydrogen via a liquid/vapor separator, not shown, the liquid product from the selective hydrogenation is fed via line 17c to Diels-Alder reactor 17d containing a fixed bed of solid maleic anhydride dispersed on silica. Reactor 17d is operated at a temperature of about from 212 to 302° F. (100° C. to 150° C.), at about atmospheric pressure and a space velocity of about 10 hr⁻¹. Space velocity and temperature is adjusted to produce a monolefin product containing less than 20 ppm of conjugated dienes. The monolefin product is then fed via line 17e to caustic scrubber 17f where it is scrubbed with 1 wt. % aqueous sodium hydroxide to remove traces of maleic anhydride and maleic anhydride-diene adduct and then passed via line 17g to stripper 17h operating at a temperature of about 392 to 572° F. (200° C. to 300° C.) to distill out water to reduce the concentration of water in the monolefin product to below 100 ppm. The monolefin product is then fed via line 19 fixed bed reactor 20 containing a catalyst bed of 0.5 wt. % rhenium on alumina catalyst. Ethylene (18) is fed via line 19a to ethenolysis reactor 22 at a mole ratio of ethylene to internal olefin of about 10. The ethenolysis reaction is operated at a temperature of about from 60° F. (16° C.) to 80° F. (27° C.) and a pressure of about 2 atm and a LHSV of about from 0.3 to 0.5 hr⁻¹ to afford about a 90% conversion of the internal olefins with an essentially 100% selectivity to NAOs. As in the case of the dehydrogenation reaction the reaction conditions may be adjusted as needed to provide the desired conversion. The reaction product is fed via line 21 to fractional distillation section 32 wherein ethylene is discharged as the overhead fraction and recycled via line 14 back to reactor 10 along with the makeup ethylene 8 fed by line 9. A C₆-C₁₄ NAO fraction is discharged by line 15, and a C₁₄-C₂₄ NAO fraction by line 16. Unreacted linear paraffins and olefins and higher NAOs are is discharged via line 23 and recycled back to the hydrotreater 5 via line 27 or fed directly to the dehydrogenation reactor 12 via line 28. (Alternatively, separate cuts can be taken from the distillation column (22) in which the higher boiling reacted paraffins and internal olefins can be recycled back to the dehydrogenation (12) by a separate line (not shown) and NAOs boiling above C₂₄ but below the higher paraffin cut can be recycled by a separate line (not shown) to the hydrotreater 5.

### EXAMPLE 2

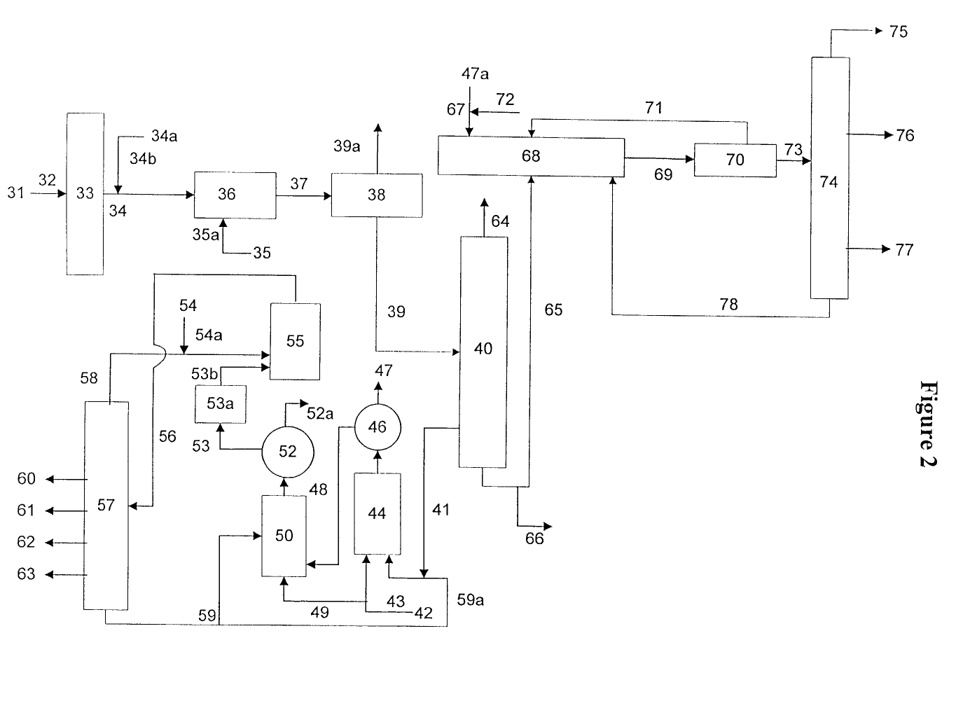

An integrated syngas, Fischer-Tropsch, ethenolysis and hydrocracking process, according to the invention will now be described with reference to FIG. 2 starting from natural gas. Natural gas 31, is fed by line 32 to scrubber operation 33 comprising an amine scrubber which removes acid gases, such as hydrogen sulfide, mercaptans and carbon dioxide followed by a sulfur scrubber containing a packed bed of zinc oxide to remove any traces of sulfur gases, e.g., hydrogen sulfide or mercaptan gases, remaining in the natural gas. The treated natural gas is fed via line 34 together with steam 34a supplied via line 34b to syngas reactor 36 where it is reacted with air or oxygen 35 provided by line 35 to effect partial oxidation of the methane. Fixed bed reactor 36 contains a methane reforming, nickel-based catalyst and is operated at a temperature between 400 and 600° C., at a pressure of between 15 and 30 bar, and at a space velocity of about 8,000 hr⁻¹ to produce a syngas containing between 1.8 and 3.5 moles of hydrogen per mole of carbon monoxide. If needed, the mole ratio of hydrogen to carbon monoxide may be adjusted by utilizing more steam, the addition of a carbon dioxide rich stream or by passing the syngas through a membrane separator (not shown).

The syngas reaction product having a mole ratio of hydrogen to carbon monoxide of about 2 is fed via line 37 to Fischer-Tropsch bubble column slurry reactor 38 containing a 12 wt. % cobalt on low density alumina catalyst having a particle size of about 25 to 65 microns and a density of about 0.4 to 7 g/cc in a 8 cs, at 100° C., synfluid slurry liquid. Prior to mixing with the slurry liquid the catalyst is reduced by contact with a 5 vol. % hydrogen, 95 vol. % nitrogen gas at about 200-250° C. for about 12 hours and then increasing the temperature to about 350-400° C. and maintaining this temperature for about 24 hours while slowly increasing the hydrogen content of the gas until the reducing gas is essentially 100% hydrogen. Reactor 38 is operated at a temperature of about from 210 to 230° C., a pressure of 25-30 bar and a synthesis gas linear velocity of about 6 to 10 cm/sec to produce a liquid hydrocarbon product boiling at about 780° F. (416° C.) containing a high proportion of C₂₆ to C₅₀ paraffins (the wax product) discharged via line 39 and a light product boiling below about 780° F. (416° C.) containing tail gases through middle distillate discharged via line 39a. Tail gases are removed from the light fraction, for example by using one or more liquid/gas separators, not shown, operating at lower temperatures and/or pressures and the remaining light product stream (condensate) comprising C₅ and higher hydrocarbons boiling below 780° F. (343° C.) optionally hydrotreated or hydroisomerized (not shown) to improve liquid fuel quality.

The F-T wax product is fed via line 39 to fractional distillation column 40 where it is fractionated into a wax fraction boiling above about 780° F. (416° C.) primarily containing C₂₆-C₅₀ linear paraffins, a high boiling bright stock fraction boiling above about 1100° F. and a primarily liquid fuel fraction boiling below about 780° F. which is taken off via line 64 and optionally hydrotreated or hydroisomerized to improve liquid fuel quality. The C₂₆-C₅₀ linear paraffin fraction is fed via line 41 to hydrotreater 44. Hydrogen (42) is furnished hydrotreater 44 via line 43 at a ratio of about 3000 SCF per Bbl of hydrocarbon feed furnished the hydrotreater. The hydrotreater is a fixed bed reactor containing a 0.5 wt. % palladium on alumina catalyst. The hydrotreater is operated at a LHSV of about from 0.5 to 1 hr⁻¹, a temperature in the range of about 700° F. to 750° F. (371° C. to 399° C.) and a pressure of about 100-120 atms. The hydrotreater hydrogenates the oxygenates, e.g., linear alcohols, and olefins in the feed to paraffins and converts any traces of organic sulfur into hydrogen sulfide. The hydrogenated reaction product is fed to liquid/vapor separator 46 where the excess hydrogen and any hydrogen sulfide is removed as the gaseous phase. Depending on the purity of the hydrogen phase it may be recycled back to the hydrotreater with makeup hydrogen or may be first passed through one or more scrubbers, not shown, before being recycled or used for other plant uses. The hydrogenated liquid phase is discharged from phase separator 46 via line 48 and fed to the dehydrogenation reactor 50 along with any recycle furnished via line 59. Hydrogen is furnished to reactor 50 via line 49 at a ratio of about 15 moles of hydrogen per mole of hydrocarbon feed including any recycle.

Dehydrogenation reactor 50 comprises a catalyst bed containing a 0.5 wt. % platinum on silicalite catalyst . The dehydrogenation reactor is initially set for operation at a reaction temperature of about 700° F. to 790° F. and a pressure of about 2 atm and at a LHSV of about 35 hr⁻¹ and the conditions then adjusted as needed give a conversion of C₂₀-C₅₀ linear paraffin to internal olefin of about 30%. The entire dehydrogenation reaction product then passes to vapor/liquid phase separator 52 wherein hydrogen and any light gases, e.g., water vapor generated by any trace oxygenates not hydrogenated in the hydrotreater, and discharged via line 52a for recycle and other plant uses after being scrubbed if needed to remove impurities. The liquid product from phase separator 52 including internal olefins and unreacted paraffins is discharged via line 53 to olefin removal opertion 53a comprising a selective hydrogenation unit, a Diels-Alder fixed bed reactor, a caustic scrubber and water stripper substantially the same as shown by items 17-17h in FIG. 1 but not shown in FIG. 2 because of space limitations. The selective hydrogenator is a fixed bed reactor containing a palladium on alumina catalyst, operated at a temperature in the range of about 392 to 437° F. (200° C. to 225° C.) a pressure of about 300 to 350 k Pag and a space velocity of about 20 hr⁻¹. Hydrogen is fed to the hydrogenation at a ratio of about 4 to 5 moles of hydrogen per mole of diene in the feed. After removal of excess hydrogen via a liquid/vapor separator, not shown, the liquid product from the selective hydrogenation is fed to a Diels-Alder reactor containing a fixed bed of solid maleic anhydride dispersed on silica. The Diels-Alder reactor is operated at a temperature of about from 212 to 302° F. (100° C. to 150° C.), about atmospheric pressure and a space velocity of about 10 hr⁻¹. Space velocity and temperature is adjusted to produce a monolefin product containing less than 20 ppm of conjugated dienes. The monolefin product is then fed to a caustic scrubber where it is scrubbed with 1 wt. % aqueous sodium hydroxide to remove traces of maleic anhydride and maleic anhydride-diene adduct and then passed to a water stripper operating at a temperature of about 392 to 572° F. (200° C. to 300° C.) to distill out water to reduce the concentration of water in the monolefin product to below 100 ppm. The monolefin product is then fed via line 53b to ethenolysis reactor 55 containing a ethenolysis catalyst bed containing a 5 wt. % tungsten on silica catalyst.. Ethylene 54 is passed to reactor 55 via line 54a along with any recycle ethylene furnished by line 58. Reactor 55 is initially operated at a reaction temperature of about 500° F. (260° C.) and a pressure of about 10 atmospheres and a liquid hourly space velocity of about 0.5 hr⁻¹ and a ethylene to internal olefins mole ratio of about 10 to 1. These conditions are then adjusted as needed to obtain an internal olefin conversion of about 90%. The selectivity of the ethenolysis reaction to NAO is about 100%.

The entire reaction mixture is then passed via line 56 to distillation section 57. Ethylene is taken off via line 58 and recycled back to reactor 55. Unreacted C₃₀-C₅₀ paraffin and NAOs above C₂₄ are taken off via line 59 and recycled back to dehydrogenation reactor 50 or depending on the olefin content to the hydrotreater, 44 via line 59a. Four normal alpha olefin fractions of varying carbon chain length and correspondingly boiling points are taken off via lines 60-63. Thus, the lower boiling C₆-C₁₀ NAOs are taken off as product fractions via line 60, C₁₁-C₁₄ NAOs via line 61, C₁₅-C₂₀ NAOs via line 62 and finally the higher boiling C₂₁-C₂₄ NAOs via line 63.

The bright stock fraction, i.e. the fraction boiling above about 1100° F. (593° C.), from distillation column 40 is fed via line 65 to hydrocracker 68 or more preferably at least a portion of the bright stock fraction is taken off via line 66 for processing as a heavy lube stock. Similarly a portion of the of the C₂₀-C₅₀ paraffin fraction from column 40 may be taken off via a separate line (not shown) for neutral lube oil processing either before or after the hydrotreater 44. (Lube oil processing involves separate hydrocracking not shown and optional hydrofinishing.) Hydrogen 47a is fed to the hydrocracker 68 via line 67.

Middle distillate hydrocracker 68 is a fixed bed reactor containing a nickel-tungsten amorphous silica-alumina catalyst and is operated at a temperature of from 650 to 850° F., a pressure of about 150 atm and a catalyst space velocity of 0.1 hr⁻¹ to 5 hr⁻¹. The reaction product from the hydrocracker is fed via line 69 to a series of vapor-liquid separators, shown in the drawing as a single box 70, to remove hydrogen from the reaction product. The hydrogen recovered from separator 70 is combined with fresh make up hydrogen 25 via lines 71-72 and recycled back to the hydrocracker or alternatively fed directly to hydrocracker 68 via line 71. The liquid hydrocrackate from the vapor liquid separators 70 is fed via line 73 to fractional distillation column 74 where it is fractionated into a fuel fraction and a lube oil fraction and taken off via lines 76 and 77 respectively. Lower boiling hydrocarbons and any residual hydrogen (tail gases) is taken off via line 75 and used as an energy source for other plant operations. The bottom fraction containing uncracked feed and other higher hydrocarbons is recycled back to the hydrocracker via line 78.

Obviously many modifications and variations of the invention described hereinabove and below can be made without departing from the essence and scope thereof.

What is claimed is:

1. A process for preparing normal alpha olefins having at least six carbon atoms which comprises the steps of:
a) dehydrogenating a hydrocarbon mixture comprising a major amount of linear paraffinic compounds containing at least ten carbon atoms under dehydrogenating reaction conditions controlled to produce a conversion of said linear paraffinic compounds to internal olefins no greater than 50 wt. % thereby minimizing the amount of dienes produced; and
b) contacting said internal olefins with ethylene under ethenolysis reaction conditions thereby producing a reaction product mixture comprising a substantial amount of normal alpha olefins having at least six carbon atoms.

2. The process according to claim 1 wherein said hydrocarbon mixture comprises at least about 70 wt. % linear paraffinic compounds having at least ten carbon atoms.

3. The process according to claim 1 wherein said hydrocarbon mixture comprises at least about 90 wt. % linear paraffinic compounds having at least ten carbon atoms.

4. The process according to claim 1 wherein a normal alpha olefin fraction having a boiling point less than the boiling point of the paraffinic fraction of said hydrocarbon mixture is recovered from the reaction product mixture of step (b) of claim 1 by distillation.

5. The process according to claim 1 wherein said hydrocarbon mixture has been prepared by Fischer-Tropsch type process.

6. The process according to claim 1 wherein said hydrocarbon mixture has been purified by extraction or adsorption prior to the dehydrogenation to remove organic sulfur compounds.

7. The process according to claim 1 wherein said hydrocarbon mixture has been hydrogenated prior to dehydrogenation to convert oxygenates and olefins to paraffins and organic sulfur compounds to hydrogen sulfide and wherein said hydrogen sulfide has been separated from said hydrocarbon mixture prior to dehydrogenation.

8. The process according to claim 1 wherein the diene content of the internal olefin feed to step b) is reduced to less than about 1 wt. %.

9. The process according to claim 8 wherein said reduction is effected by selective diene hydrogenation.

10. The process according to claim 8 wherein said reduction is effected by selective diene adsorption.

11. The process according to claim 1 wherein conjugated dienes are removed from the internal olefin feed to step b) by reacting said conjugated dienes with a physically separable dienophile to produce an adduct of the conjugated diene and the dienophile and separating said adduct and unreacted dienophile from said internal olefin feed.

12. The process of claim 11 wherein said dienophile is maleic anhydride.

13. The process of claim 12 wherein said maleic anhydride is dispersed on an inorganic support and said adduct deposits out on said support.

14. The process of claim 12 wherein said reaction is conducted as a liquid:liquid reaction using molten maleic anhydride as one immiscible liquid phase and said internal olefin phase as the other and wherein said molten maleic anhydride together with the adduct are separated from said internal olefin phase.

15. The process according to claim 12 wherein said reaction is conducted as a liquid:liquid phase reaction using said internal olefin feed is one phase and maleic anhydride dissolved in an immiscible inert organic solvent as the other phase and wherein following reaction the resulting adduct and excess maleic anhydride are removed with the immiscible solvent from said internal olefin liquid phase.

16. The process of claim 8 wherein following reduction of the diene content to below 1 wt. %, conjugated dienes are reduced to below 100 ppm by reaction with a physically separable dienophile to produce an adduct and separating said adduct and unreacted dienophile from said internal olefin feed.

17. The process of claim 16 wherein said dienophile is maleic anhydride.

18. The process of claim 17 wherein said maleic anhydride is dispersed on an inorganic support and said adduct deposits out on said support.

19. The process of claim 17 wherein said reaction is conducted as a liquid:liquid reaction using molten maleic anhydride as one immiscible liquid phase and said internal olefin phase as the other and wherein said mollen maleic anhydride together with the adduct are separated from said internal olefin phase.

20. The process according to claim 17 wherein said reaction is conducted as a liquid:liquid phase reaction using said internal olefin feed as one phase and maleic anhydride dissolved in an immiscible inert organic solvent as the other phase and wherein following reaction resulting adduct, excess maleic anhydride are removed with the immiscible solvent from said internal olefin phase.

21. The process according to claim 1 wherein the normal alpha olefins are separated from the ethenolysis reaction product and the remaining paraffins and internal olefins are recycled to the dehydrogenation step.

22. The process according to claim 6 wherein normal alpha olefins are separated from the ethenolysis reaction product and remaining paraffins and internal olefins are recycled to the purification step preceding the dehydrogenation step.

23. The process of claim 1 wherein a normal alpha olefin product is recovered from said reaction product mixture have an average molecular carbon atom number within about 25 carbon atoms of the linear paraffinic fraction of said hydrocarbon fraction.

24. The process of claim 1 wherein said dehydrogenation is conducted at temperatures in the range of about from 500° F. to 900° F., pressures in the range of about from 0.5 to 3 atms, and liquid hourly space velocity in the range of about from 1 to 50 hr⁻¹.

25. The process of claim 1 wherein said dehydrogenation is conducted in the presence of a catalyst comprising at least one metal selected from the group of Group VIII noble metal.

26. The process of claim 1 wherein said ethenolysis is conducted at temperatures in the range of about from 50° F. to 600° F., pressures in the range of about from 1 to 15 atms and space velocities in the range of about from 0.1 to 10 hr⁻¹.

27. The process of claim 1 wherein said ethenolysis is conducted in the presence of a catalyst comprising ruthenium at temperatures in the range of about from 60° F. to 80° F., pressures in the range of about from 1.5 to 3 and space velocities in the range of about from 0.2 to 2 hr⁻¹.

28. The process of claim 1 wherein said ethenolysis is conducted at temperatures in the presence of a catalyst comprising tungsten in the range of about from 400° F. to 600° F., pressures in the range of about from 8 to 12 atms and space velocities in the range of about from 0.2 to 2 hr⁻¹.

29. A process for upgrading a Fischer-Tropsch type reaction product containing at least 70 wt. % C₁₆-C₁₀₀ linear paraffinic compounds, into lower boiling NAOs which comprises the steps of:
a) dehydrogenating said Fischer-Tropsch reaction product to produce C₁₆-C₁₀₀ linear internal olefins under dehydrogenating conditions adjusted to produce a conversion based on said linear paraffinic compounds of about from 15 to 50 wt. %.
b) contacting said C₁₆-C₅₀ linear internal olefins with ethylene in the presence of an ethenolysis catalyst under reactive conditions thereby producing a reaction product comprising a substantial amount of lower boiling NAOs.

30. The process of claim 29 wherein Fischer-Tropsch type reaction product is a Fischer-Tropsch reaction product.

31. The process of claim 29 wherein said Fischer-Tropsch type reaction product is a Kobbel-Englehardt reaction product.

32. An integrated ethenolysis and hydrotreating or hydroisomerization process for upgrading Fischer-Tropsch type hydrocarbon reaction products containing at least about 20 wt. % C₁₆-C₅₀ linear paraffinic compounds into C₆-C₂₄ NAOs and at least one liquid fuel or base oil which comprises the steps of:
a) fractionating said Fischer-Tropsch type product to produce a wax fraction comprising at least 70 wt. % linear C₁₆-C₅₀ linear paraffinic compounds and at least one other fraction boiling at a temperature range different than said wax fraction;
b) dehydrogenating the wax fraction of step (a) to produce C₁₆-C₅₀ linear internal olefins and wherein said dehydrogenation is conducted at a conversion no greater than 50 wt. % based on said wax fraction;
c) contacting said C₁₆-C₅₀ linear internal olefins with ethylene in the presence of an ethenolysis catalyst under reactive condition thereby producing a reaction product comprising a substantial amount of C₆-C₂₄ NAOs; and
d) hydrotreating or hydroisomerization at least one of said other fractions of step (a) and fractionating the resulting effluent and recovering at least one liquid fuel fraction or at least one lubricating oil fraction.

33. A process for converting C₁-C₃ alkane gasses into C₆ and higher NAOs which comprises the steps of:
a) reforming said C₁-C₃ alkanes into synthesis gas;
b) contacting said synthesis gas with a Fischer-Tropsch catalyst under reactive conditions to yield two hydrocarbon product streams, one a wax containing product boiling above a selected value in the range of about 350° F. (177° C.) to about 700° F. (371° C.) comprising at least 20 wt. % C₁₀-C₅₀ linear paraffinic compounds, and a second hydrocarbon product boiling below about said value, containing hydrocarbons boiling in the liquid fuel range;
c) fractionating the wax containing product of step (b) into fractions comprising at least a wax fraction comprising at least 70 wt. % C₁₀-C₅₀ linear paraffinic compounds, and a heavy fraction boiling above about 1100° F. (393° C.);
d) dehydrogenating the wax fraction of step (a) at a conversion, based on said linear paraffinic compounds, no greater than 50 wt. % to produce C₁₀-C₅₀ linear internal olefins;
e) contacting said C₁₀-C₅₀ linear internal olefins with ethylene in the presence of an ethenolysis catalyst under reactive conditions thereby producing a reaction product comprising a substantial amount of C₆ and higher NAOs; and
f) separating said reaction product of step (e) to recover at least one NAO fraction within the range of C₆ and higher NAOs having a NAO purity of at least 70 wt. %.

34. A process for converting C₁-C₃ alkanes into C₆-C₂₄ NAOs which comprises the steps of:
a) reforming said C₁-C₃ alkanes into synthesis gas;
b) contacting said synthesis gas with a Fischer-Tropsch catalyst under reactive conditions to yield a reaction product mixture of hydrocarbons comprising C₁₀-C₅₀ paraffinic compounds, vacuum gas oil, middle distillate, gasoline light oxygenates and light olefins;
c) fractionating the Fischer-Tropsch reaction product mixture of step (b) into separate fractions comprising a wax fraction containing at least 70 wt. % linear C₁₆-C₅₀ paraffinic compounds at least one liquid fuel fraction and at least one higher boiling fraction boiling above the temperature of the wax fraction;
d) dehydrogenating the wax fraction of step (c) at a conversion no greater than about 50 wt. %, based on said linear paraffinic compounds, to produce C₁₆-C₅₀ linear internal olefins;
e) contacting said C₁₆-C₅₀ linear internal olefins with ethylene in the presence of an ethenolysis catalyst under reactive conditions thereby producing a reaction product comprising a substantial amount of C₆-C₂₄ NAOs;
f) fractionating the reaction product of step (e) into at least one NAO fraction within the range of C₆-C₂₄ having a C₆-C₂₄ NAO purity of at least 70 wt. % and a higher boiling fraction containing NAOs having more than 20 carbon atoms and branched olefins and paraffins; and
g) hydrotreating or hydroisomerizing at least one of the liquid fuel fractions and higher boiling fractions recovered in step (c) and the higher boiling fraction recovered in step (f) to produce a reaction product comprising liquid fuel hydrocarbons.

35. An NAO mixture made by the process according to claim 1.
